(19) United States
(12) Patent Application Publication
(10) Pub. No.: US 2019/0083192 A1
(43) Pub. Date: Mar. 21, 2019

(54) DEVICES INCORPORATING A MAGNET GUIDED FLEXIBLE REGION AND METHODS OF USE THEREOF

(71) Applicant: COOK MEDICAL TECHNOLOGIES LLC, Bloomington, IN (US)

(72) Inventors: Ralf Spindler, Solsberry, IN (US); Davorin Kevin Skender, Bloomington, IN (US); Patrick J. Zito, St. Louis Park, MN (US)

(73) Assignee: COOK MEDICAL TECHNOLOGIES LLC, BLOOMINGTON, IN (US)

(21) Appl. No.: 16/130,097

(22) Filed: Sep. 13, 2018

Related U.S. Application Data
(60) Provisional application No. 62/560,481, filed on Sep. 19, 2017.

(51) Int. Cl. A61B 34/00; H01F 7/02; A61F 2/95; A61B 17/12; A61L 31/14; A61L 31/02; A61L 31/04; A61L 31/06

(52) U.S. Cl. CPC A61B 34/73; H01F 7/02; A61F 2/95; A61B 17/1214; A61L 31/14; A61F 2002/048; A61L 31/048; A61L 31/049; A61L 31/06; A61L 31/041; A61L 31/022

(57) ABSTRACT

One aspect of the present invention relates generally to the field of medical devices, and more particularly, to a medical device including an elongated flexible region. In one embodiment, a magnetic assembly is coupled to a surface of the elongated flexible region and provides a stabilization force to maintain the elongated flexible region in a preferred configuration. In one embodiment, the flexible region is a region having a stacked coil configuration and a straightened configuration.

### RELATED APPLICATIONS

This non-provisional patent application claims priority to U.S. Provisional Patent Application No. 62/560,481, filed Sep. 19, 2017, the contents of which are incorporated by reference in their entirety.

### TECHNICAL FIELD

One aspect of the present invention relates generally to the field of medical devices, and more particularly, to a medical device including an elongated flexible region. A magnetic assembly is coupled the elongated flexible region and provides a stabilization force to maintain the elongated flexible region in a preferred configuration without entanglement or knot formation. In one embodiment, the flexible region is a region having a stacked coil configuration and a straightened configuration.

### BACKGROUND

Medical devices like coils, catheters, and wires often need to follow specific conformational limitations to stay fully functional. For some such devices there may be a limitation of bending, they should not be kinked, or they should not form a knot during in vivo placement. In the treatment of urinary diseases where urine is not sufficient removed from the body (Hydronephrosis) artificial drainage methods are used. There, renal stents with pigtail ends are sometimes placed to allow the device fixation and the drainage of urine.

Renal stents like the Cook Universal® Firm Ureteral Stent (Cook Medical Inc., Bloomington, Ind.) are commonly removed by metal claws which grasp the stents. Another commonly used method for retrieval involves the patient or physician is pulling the device manually out by hand, often with the assistance of a string that attaches to the device and extends from the body of the patient. However, if the device is stuck inside the organ, for example, if a pigtail has interloped inside the kidney, then, such methods cannot be used. A surgical intervention may then be required.

Magnets with complex magnetic patterns which can be produced and even 3D printed on ferromagnetic materials. For example, the magnetic pattern can be printed on these materials by using a specialized 3D-like printer which converts Pixels into Maxels (magnetic Pixel). Special magnetic patterns may be printed on ferromagnetic materials which are then attached to incorporated into a coil, a catheter, a snare, a wire or other devices.

### SUMMARY

One aspect of the present invention provides a medical device including an elongated flexible body having a stacked coil configuration and a straightened configuration. A magnetic assembly is coupled to a surface of the elongated flexible body and has a magnetic polarity profile providing a stabilization force to maintain the elongated flexible body in a stacked coil configuration. The stacked coil configuration may include, for example, at least two, or three, or more complete coils. In various embodiments, the medical device is a stent, an ureteral stent, a coil, a snare, a wire guide or a catheter. In a preferred embodiment, the medical device is an ureteral stent.

In one embodiment, the magnetic assembly includes a plurality of magnets, each having a surface and a plurality of north and south poles aligned along the surface, where the positioning and magnetic polarity profile of the plurality of magnets is such that they provide a stabilization magnetic force to maintain the elongated flexible body in the stacked coil configuration and at least reduce the possibility of entanglement or knot formation.

In one embodiment, the magnetic assembly includes a plurality of magnets, each having a plurality of north and south poles aligned along an axis of elongation of the flexible body. The positioning and magnetic polarity profile of the magnets is such that they provide a stabilization magnetic force to maintain the elongated flexible body in the stacked coil configuration.

In another embodiment, the linear arrangement of north and south poles of at least two of the magnets is paired to provide a localized attractive force between the magnets. In yet another embodiment, the linear arrangement of north and south poles of at least two of the magnets is paired to provide a localized repulsive fore between the magnets.

The linear arrangement of north and south poles may be such that the elongated body is biased towards the stacked coil configuration. In one embodiment, the strength and linear arrangement of the north and south poles is such that, when in the stacked coil configuration, surfaces of the stacked coil are not in contact. In another embodiment, the linear arrangement of north and south poles at least reduces interloping of the elongated flexible body.

The elongated flexible body may include a polymer. In such embodiments, the magnetic assembly may be at least partially imbedded in the polymer. In another embodiment, the magnetic assembly includes samarium-cobalt, neodymium-iron-boron, or a combination of these materials. The magnetic assembly may be coupled the surface by 3-D printing. In another embodiment, the radial dimension of the magnetic assembly varies along the length of the elongated body.

Another embodiment of the present invention provides a uretheral stent including at least one pigtail tip having a magnetic assembly coupled to its surface. The magnetic polarity of the magnetic assembly provides a stabilization force to maintain the pigtail tip in a stacked coil configuration without interlooping, The magnetic assembly includes a linear arrangement of north and south poles, aligned with an elongation axis of the pigtail tip, and includes samarium-cobalt, neodymium-iron-boron, or a combination of these materials.

### BRIEF DESCRIPTION OF THE DRAWINGS

FIGS. 1(A) and 1(B) are a schematic illustrations of the magnetic field strength in the vicinity of a conventional magnet (FIG. 1(A)) and a magnet having a plurality of north and south poles (FIG. 1(B)).

FIGS. 2(A) and 2(B) are schematic illustrations of part of a stent having a pig-tail tip. FIG. 2(A) illustrates the pigtail in a stacked coil configuration. FIG. 2(B) is an enlarged view showing the positioning of an magnetic assembly on a surface of the pigtail region of the catheter.

### DETAILED DESCRIPTION OF THE PREFERRED EMBODIMENTS

### Definitions

Unless otherwise defined, all technical and scientific terms used herein have the same meaning as commonly understood by one of ordinary skill in the art to which this invention pertains. In case of conflict, the present document, including definitions, will control. Preferred devices, methods and materials are described below, although devices, methods and materials similar or equivalent to those described herein can be used in the practice of the present invention. All publications, patent applications, patents and other references mentioned herein are incorporated by reference in their entirety.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

As used herein, the term “body cavity” means any body passage or lumen, including but not limited to vascular coronary or peripheral vessels, esophageal, intestinal, biliary, bronchial, reproductive, urethral and ureteral passages. The term also encompasses a body passage formed during a surgical procedure.

### Devices Incorporating a Magnet Guided Flexible Region

One aspect of the present invention provides a medical device having a magnetic assembly coupled to at least one surface. Preferably, the medical device includes a flexible region and the magnetic assembly is positioned on or in the flexible region such that the resultant magnetic force from the magnetic assembly acts to bias the flexible region in a preferred configuration and to maintain the flexible region in this configuration.

In one embodiment, the magnetic assembly includes a magnetic material having a plurality of north and south poles aligned in the defined configuration on the surface. The magnetic material may include an suitable ferromagnetic material, such as iron, cobalt or nickel. In preferred embodiments the magnetic material is in the form of a rare-earth magnet formed from samarium-cobalt or neodymium-iron-boron. The magnetic material may also be in the form of individual magnetizable particles embedded into a polymeric film. For example, the magnet be a flexible magnet, such as those available form Magnum Magnetics Corporation, Marietta, Ohio 45750 USA.

The magnetic assembly may be formed from a plurality of discrete magnets that are bonded together. In another embodiment, the magnetic structure contains patterns of north and south poles formed on a single piece of magnetizable material. Here, the resulting magnetic field of these patterns of north and south poles define the magnetic field in the vicinity of the magnet. In one preferred embodiment, the magnetic assembly is formed by 3-D printing magnetic particles onto a surface of the device to provide a desired sequence of north and south poles. In some embodiments, there may be a continuous line of magnetic poles printed on the surface with the same polarity.

In another embodiment, the magnetic assembly is in the form of a “polymagnet”, such as those available from Correlated Magnetics Research LLC, Huntsville, Ala. 3580, or those formed as described in U.S. Patent Application Publication Number 20160365187, entitled “SYSTEM AND METHOD FOR PRODUCING MAGNETIC STRUCTURES”, the contents of which are hereby incorporated by reference.

In various embodiments, the magnetic pole size may be between, for example, 3 mm and 0.5 microns, or 3 mm and 1.0 micron, or 3 mm and 10 microns, or 3 mm and 100 microns, or 3 mm and 1 mm, 1 mm and 0.5 microns, or 0.5 mm and 0.5 microns, or 0.1 mm and 0.5 microns. Typical field strength at the magnet surface may be between, for example 100 gauss and 500 gauss, or 200 gauss and 500 gauss, or 300 gauss and 500 gauss, or 400 gauss and 500 gauss.

Where the device, for example a wire guide, is formed from a magnetizable material, the magnetic assembly may be formed within the material of the device itself. In other embodiments, the flexible region of the device may be formed from, or incorporate, a polymer. For example, the flexible region may include a nylon, polyamide, polyolefin, polyester, polyurethane, fluoropolymer, polyethylene, polytetrafluoroethylene (PTFE), polyethyleneterepthalate (PET), polyvinyl chloride, latex, natural rubber, synthetic rubber, elastomer, silicone or mixtures or copolymers of at least two of these materials. In such embodiments, the magnetic assembly may be at least partially, or completely, imbedded in the polymer.

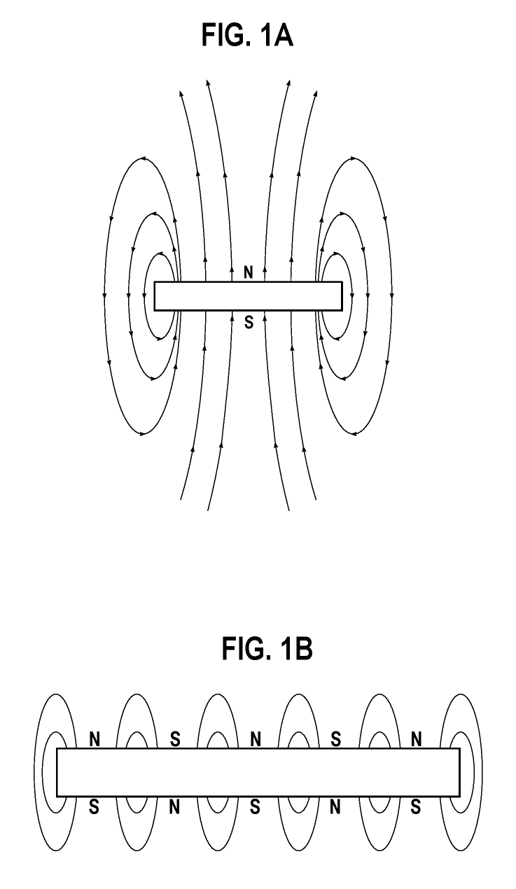

The patterns of north and south poles on the surface of the magnetic assembly may be selected to produce a tightly focused magnetic field in the vicinity of the surface. Turning now to FIG. 1. FIG. 1(A) illustrates the magnetic field around a conventional magnet, whereas FIG. 1(B) shows the magnet field around a magnet having a plurality of alternating north and south magnetic poles. The field of the magnet with alternating poles is much more focused than is that of the conventional magnet, resulting in a higher field strength in the region close to the surface. As a result, when a magnetizable or magnetic material is placed close to the alternating poles, the material is held tighter than will the conventional magnet when the material is placed at the same separation distance.

However, at greater separation distances, the field of the conventional magnet is stronger than that of the alternating pole magnet. As a result, at greater distances, this magnet will exhibit less of an attractive force on an magnetizable material than will the conventional magnet. The force exerted by either magnet will cause the magnetizable material to be drawn towards the magnet. However, because the conventional magnet exerts a greater force at larger distances, the magnetizable material is more likely to be accelerated towards the magnet. With the alternating pole magnet, this will not happen until the two are closer together. The use of the alternating pole magnet allows the force profile to be tuned such that a desired pull on the magnetizable material is obtained. By selecting the configuration of magnetic poles and the strength of the individual poles, the engagement distance and holding strength may be selected in a controlled way.

An alternating pole magnet can be paired with another such magnet such that the two magnets to attract or repel each other in a controlled way. In one embodiment, two such magnets are attached to different portions of a flexible member. By varying the strength and positioning of the individual poles, the magnet can be made to align and attach to each other when they approach within a certain distance. This allows the two magnets to be aligned with each other in a preferred configuration.

In another embodiment a series of alternating pole magnets are positioned a different positions along the surface of a flexible device. The strength and positioning of the individual poles of each of the magnets may be chosen so that different portions of the surface align to form a preferred configuration. In some embodiments, the surface may include pairs of magnets that attract and other pairs that repel each other. The overall configuration of the magnets may be chosen so that the resultant magnet force is such that, in an equilibrium position, the surfaces are aligned as required but do not touch each other. Instead, two regions of the flexible body are held at the equilibrium position at a selected distance apart.

In another embodiment, the positioning of the magnets may be such that the resultant magnet field, together with mechanical bending forces present within the device prevent entanglement of the flexible region or knot formation. For example, in the case of a pig-tailed tipped catheter, the resultant magnet force may prevent the formation of knots and stabilize the tip in the stacked coil configuration. The magnetic assembly may be designed in such a way to provide optimum device flexibility and mechanical strength (compensation of moments along the length of the pig-tail).

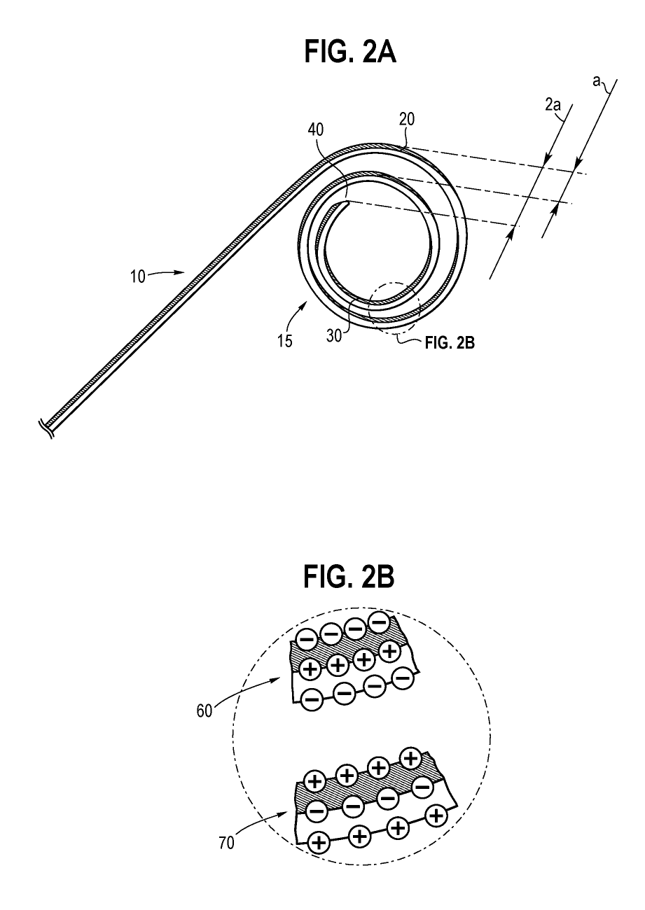

The present invention will now be illustrated with reference to a stent having a coiled pig-tail tip, such as the tip illustrated in FIG. 2(A). However, the invention also encompasses other devices with a flexible region having a preferred configured configuration of the region surfaces with respect of each other. Examples of such devices include, but are not limited to, stents, uretheral stents, catheters, coils, snares, wire guides. In a preferred embodiment, the device is a uretheral stent having at least one pig-tailed tip.

Turning first to FIG. 2(A), there is here illustrated part of a stent 10 having a pigtail tip 15. Pigtail tip 15 includes two coils, inner coil 30 and outer coil 20, shown here in a stacked coil configuration. In various other embodiments, the pig-tailed tip may include 1, 2, 3, 4 or more coils in the stacked coil configuration shown in FIG. 2(A). The tip is formed of a flexible material and is normally biased to assume the stacked coil configuration. However, the tip may assume a straightened configuration for positioning the device within a body cavity of a patient and for removal of the device. A longitudinal axis of the device extends along the elongated stent 10 to the end 40 of pig-tailed tip 15.

A magnetic assembly is coupled to a surface of the elongated flexible body of the pig-tailed tip. The surface of the assembly includes magnets including a plurality of magnetic north and south poles having a resultant magnetic polarity profile providing a stabilization force to help maintain the tip in the stacked coil configuration and avoid entanglement of the pig-tail and the possibility of knot formation. The magnets may be any of the multipole magnets disclosed herein. Such entanglement, if it occurs within the kidney of other body lumen may prevent the withdrawal of the device from the patent, and possibly require surgical retrieval.

In one embodiment, the north and south poles are aligned along an axis of elongation (longitudinal axis) of the tip. The positioning and polarity profile of the plurality of magnets is such that they provide a stabilization magnetic force to maintain the elongated flexible body in the stacked coil configuration without entanglement of the pigtail.

In one embodiment, the linear arrangement of north and south poles in at least two of the regions of the magnetic assembly is paired to provide a localized attractive force between the regions. The linear arrangement of north and south poles in at least two of the regions may also to paired to provide a localized repulsive force between these regions. In one embodiment, when the pigtail is in the stacked coil configuration, the resultant magnetic force resulting from the attractive and repulsive magnetic forces results in the coils taking up the stacked coil configuration without the surfaces of the coils touching the surfaces of neighboring coils. Such a configuration is illustrated in FIG. 2(A). Here, outer coil 20 and inner coil 30 are positioned so as to maintain a separation “a” between the coils of the pigtail.

### Methods of Using Devices Incorporating a Magnet Guided Flexible Region

Uses and advantages of the devices disclosed herein will now be illustrated with particular reference to double pigtail uretheral stent. A uretheral stent is a soft tube, typically about 10-12 inches long having a pigtail tip at each end. The stent is placed in the ureter to assist drainage of urine from the kidney to the bladder. When implanted within the body of a patient, one end (pigtail) of the stent sits inside the kidney, and one end (pigtail) sits in the bladder. The tips assume a stacked coil configuration and stabilizing the position of the stent and preventing its dislodgement.

Typically, the stent is positioned in the ureter using a cystoscope. The stent is passed through the urethra until one end enters the kidney and the other end remains in the bladder. The pig-tail tips at both ends of the stent assume their stacked coil configurations when released from the cystoscope and prevent the stent from shifting or being expelled from the body of the patient.

The stent will eventfully be removed from the urinary system of the patient. Stents often have a thread, used for removal, that passes through the urethra and remains outside the body. Stents with a thread may be removed by pulling on the thread. Stents without a thread may be removed using a cystoscope.

Complications may arise if the stacked coils of either of the pigtail tips become entangled or knotted. In such situations, a surgical intervention may be required to remove the stent. Devices as disclosed therein reduce the possibility of knot formation by properly positioning the pigtail coils with respect to each other.

The devices as disclosed herein also have applications in other procedures including the use of devices having coiled “pigtail” like tips. Such devices include pancreatic stents and biliary stents, as well as devices utilized for pleural drainage, bronchoscopic drainage, transrectal drainage, thoracostomy, percutaneous abdominal drainage, pelvic drainage and abscess drainage.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

What is claimed is:

1. A medical device comprising:
an elongated flexible body having an axis of elongation, the elongated flexible body having a stacked coil configuration and a straightened configuration,
a magnetic assembly coupled a surface of the elongated flexible body and having a polarity profile providing a stabilization force to maintain the elongated flexible body in the stacked coil configuration.

2. The medical device of claim 1, wherein the stacked coil configuration comprises at least two complete coils

3. The medical device of claim 2, wherein the stacked coil configuration comprises at least three complete coils.

4. The medical device of claim 1, wherein the medical device is selected from the group consisting of a stent, an uretheral stent, a coil, a snare, a wire guide and a catheter.

5. The medical device of claim 4, wherein the medical device is an uretheral stent.

6. The medical device of claim 1, wherein the magnetic assembly comprises a plurality of magnets, each having a surface and a plurality of north and south poles aligned along the surface, wherein the positioning and magnetic polarity profile of the plurality of magnets is such that they provide a stabilization magnetic force to maintain the elongated flexible body in the stacked coil configuration.

7. The medical device of claim 1, wherein the magnetic assembly comprises a plurality of magnets each comprising a linear arrangement of north and south poles, wherein the plurality of magnets is aligned such that the linear arrangement of north and south poles aligns with the axis of elongation of the elongated flexible body.

8. The medical device of claim 7, wherein the linear arrangement of north and south poles in at least two of the magnets is paired to provide a localized attractive force between the at least two of the magnets.

9. The medical device of claim 7, wherein the linear arrangement of north and south poles in at least two of the magnets is paired to provide a localized repulsive fore between the at least two of the magnets.

10. The medical device of claim 1, wherein the magnetic assembly comprises a linear arrangement of north and south poles extending along an axis of elongation, wherein the linear arrangement of north and south poles is such that the elongated body is biased towards the stacked coil configuration.

11. The medical device of claim 10, wherein the strength and linear arrangement of the north and south poles is such that, when in the stacked coil configuration, surfaces of the stacked coils are not in contact.

12. The medical device of claim 10, wherein the linear arrangement of north and south poles at least reduces interloping of the elongated flexible body.

13. The medical device of claim 1, wherein the elongated flexible body comprises a polymer.

14. The medical device of claim 13, wherein the magnetic assembly is at least partially imbedded in the polymer.

15. The medical device of claim 1, wherein the magnetic assembly comprises a material selected from the group consisting of samarium-cobalt, neodymium-iron-boron, and a combination thereof.

16. The medical device of claim 1, wherein the magnetic assembly coupled the surface is applied by 3-D printing.

17. The medical device of claim 1, wherein a radial dimension of the magnetic assembly varies along a length of the elongated body.

18. A uretheral stent comprising:
a pigtail tip;
a magnetic assembly coupled a surface of the pigtail tip and having a magnetic polarity profile providing a stabilization force to maintain the pigtail tip in a stacked coil configuration without interlooping,
wherein the magnetic assembly comprises a linear arrangement of north and south poles, aligned such that the linear arrangement of north and south poles aligns with an elongation axis of the pigtail tip, and wherein the magnetic assembly comprises a material selected from the group consisting of samarium-cobalt, neodymium-iron-boron, and a combination thereof.

19. The uretheral stent of claim 18, wherein the, wherein the magnetic assembly coupled the surface is applied by 3-D printing
